(19) United States
(12) Patent Application Publication
(10) Pub. No.: US 2024/0382535 A1
(43) Pub. Date: Nov. 21, 2024

(54) Use of Sustainable, Modified and Enhanced Aquaculture Limulus Amebocyte Lysate Protein and Hemolymph Compounds as a Biologic Broad Spectrum Antimicrobial Therapy.

(71) Applicants: Anthony Lee Dellinger, Burlington, NC (US); Rachel Tinker-Kulberg, Hillsborough, NC (US); Terry Earl Brady, The Valley (AI); Melinda K. M. Goddard, The Valley (AI); Christopher L. Kepley, Montvale, VA (US); Lance Toland, Griffin, GA (US)

(72) Inventors: Anthony Lee Dellinger, Burlington, NC (US); Rachel Tinker-Kulberg, Hillsborough, NC (US); Terry Earl Brady, The Valley (AI); Melinda K. M. Goddard, The Valley (AI); Christopher L. Kepley, Montvale, VA (US); Lance Toland, Griffin, GA (US)

(21) Appl. No.: 18/198,312

(22) Filed: May 17, 2023

(51) Int. Cl. A61K 35/612; C07K 14/435

(52) U.S. Cl. CPC A61K 35/612; C07K 14/43509

(57) ABSTRACT

This invention relates to a broad-spectrum antimicrobial marine biologic composition for bloodstream infections, derived from smeLAL and optional hemolymph of sustainable aquaculture horseshoe crabs. The formulation serves as an injectable treatment with broad-spectrum antimicrobial activity and an array of therapeutic mechanisms. In one embodiment, subject to clinical studies and characterization of cross-species immunogenicity, the composition may act as a breakthrough antimicrobial—or as a last-resort therapy for critically ill sepsis patients. In an alternative embodiment, the biologically derived antimicrobial agent represents a viable primary therapy with advantages over synthetic antibiotics, including a rapid therapeutic window. Given unmatched efficacy, an adjuvant smeLAL immunoglobulin therapy may be warranted to allow routine use if needed to address cross-species immunogenicity. The invention can thus be administered through various methods, including infusion, subcutaneous injection, topical application, oral, or other delivery techniques, offering potential utility against antimicrobial-resistant pathogens and mutated infectious strains.

### CROSS REFERENCE TO RELATED APPLICATION(S)

This application is a continuation-in-part of U.S. patent application Ser. No. 18/131,558, titled “USE OF A SUSTAINABLE. MODIFIED AND ENHANCED AQUACULTURE LIMULUS AMEBOCYTE LYSATE PROTEIN FOR DETECTION AND CHARACTERIZATION OF INFECTIOUS PATHOGENS IN BIOLOGIC SAMPLES FOR PATIENT SCREENING, DIAGNOSIS AND THERAPEUTIC MANAGEMENT,” filed on Apr. 6, 2023.

### STATEMENT REGARDING FEDERALLY SPONSORED RESEARCH OR DEVELOPMENT

The invention was made with government support under Grant Nos. 1819562, 2101278 and 2212920 awarded by the National Science Foundation. The US government does not have rights to the invention.

### FIELD OF THE INVENTION

The present invention relates to the field of broad-spectrum antimicrobial therapeutics, in particular, a biologic comprising sustainable, modified and enhanced Limulus Amebocyte Lysate (smeLAL), hemolymph and biomolecules derived from aquaculture horseshoe crabs. In the primary embodiment of the invention and upon successful clinical studies, the marine-derived biological is a novel, injectable antimicrobial agent effective against diverse classes of pathogens, including resistant strains. Whereupon, expected efficacy may nonetheless warrant use as a therapy of last resort in the event of significant cross-species immunogenicity, particularly in severe cases of sepsis. Alternatively, development of an adjuvant therapy employing smeLAL immunoglobulin may likewise be warranted if breakthrough therapeutic efficacy is demonstrated to allow routine administration in life-saving treatment of resistant pathogens. This sustainable aquaculture biologic agent has been shown to neutralize gram-negative bacteria, gram-positive bacteria, fungal, viral, combinations thereof and infectious pathogens of unknown origin. Likewise, given expected success in clinical studies, an array of alternative embodiments may include topical and ingestible administration of these molecules, as well as injectable formulations. The achievement of the marine-derived biologic composition is directly related to production of smeLAL using controlled aquaculture operations that overcome wild capture substrate inconsistencies, batch-to-batch variabilities, species sustainability and supply chain limitations.

### BACKGROUND

A recent invention disclosure taught a diagnostic assay comprising a sustainable, modified, and enhanced Limulus Amebocyte Lysate (smeLAL) derived from aquaculture horseshoe crabs and methods thereof. The smeLAL diagnostic enables rapid screening of asymptomatic patients as well as those with suspected microbial infections using highly sensitive and specific direct pathogen detection and simultaneous antibiotic susceptibility testing (AST) in a biological specimen (irrespective of sample opacity). The parent application described manifold improvements over existing blood stream diagnostic methods. Advantages include faster results (turn-around-time), low-volume specimen capabilities, high pathogen sensitivity and specificity, and compatibility with complex patient specimens.

This filing is a continuation-in-part of U.S. patent application Ser. No. 18/131,558 with expanded aquaculture-derived smeLAL utility beyond diagnostic testing. The present invention consists of an aquaculture-derived sustainable, modified, and enhanced marine biologic consisting of purified immunity constituents contained in the cellular material and hemolymph of aquaculture horseshoe crabs. Disclosed herein, the marine-derived therapeutic contains biologically active, native defense molecules that include, but are not limited to, proteins, antimicrobial factors, peptides, granule amebocytes and intracellular components thereof, as well as hemocyanin. The proposed biologic invention for use as a direct interventional antimicrobial therapy leverages sustainable aquaculture production and consistency of horseshoe crab immunology components that have enabled species survival and protection from a diverse range of pathogens for millennia.

Sepsis is a major public health and economic concern that results in one death every 2.8 seconds, worldwide [Sepsis Alliance, 2018]. In the United States, sepsis contributes to 270.000 deaths per year [Ferreira et al, 2022], is the leading cost to hospitals [Paoli et al., 2018], and is the primary cause of readmissions [Fingar, 2015]. Globally, sepsis results in 11 million deaths and affects 50 million people each year [Angus et al., 2001; Kumar et al., 2011; Rudd et al., 2020]. Early and accurate detection of pathogens in a biological specimen, prior to onset of sepsis, can mitigate tissue and organ damage, avert mortality, and reduce associated hospital costs [Evans et al., 2021]. Without rapid detection and appropriate antibiotic administration, the risk of patient mortality increases by 7.6% per hour [Kumar et al., 2006].

Despite medical advances, sepsis is often fatal [Angus et al., 2006]. Most who survive it nonetheless suffer from permanent organ damage [Chang et al., 2010], cognitive impairment and/or physical disability [Iwashyna et al., 2010]. The dearth of rapid and accurate diagnostic tests for pathogens in biological specimens has hampered the accuracy and timeliness of clinical decision making, thus propelling sepsis to become the leading cause of premature death worldwide [CDC, 2017]. Lacking timely, accurate detection tools, physicians often rely on indirect assessments of confounding symptoms and clinical pragmatism to initiate treatment within one hour [Buckman et al., 2018]. Consequently, untenable rates of morbidity and mortality persist in a pathogenic era with intensifying risks from antibiotic microbial resistance (AMR) due to unilateral administration of broad-spectrum therapeutics, often compounded by suboptimal drug selection for unknown pathogens and/or patient compliance issues that fail to eradicate otherwise susceptible organisms before resistant mutations occur [Hsu, 2020].

With regard to other prospective detection methods, wild-capture Limulus Amebocyte Lysate (LAL) from horseshoe crabs (Limulus polyphemus) was shown to be the most sensitive (parts-per-trillion), reliable, rapid (1-hour reaction time), and easiest method for bacterial endotoxin testing (BET) by the 1970s. LAL specificity derives from its two distinct Factor C and Factor G pathways. The Factor C pathway is specific to lipopolysaccharide (LPS), unique to the surface of gram-negative bacteria; and the Factor G pathway, which is specific to 1-3-β-D-Glucan (βDG) and represents one of the most abundant cell wall components of pathogenic fungi. Once given approval by the FDA, use of LAL in BET sterility testing for injectable drug and implanted device quality control was widely adopted in industry; whereas constituent complexity and interfering substances in human specimens and inconsistent wild-capture LAL performance precluded its clinical use due to unreliability, as reported in research efforts that spanned decades.

Various specimen manipulation strategies have been applied and evaluated to overcome biological compatibility issues including: specimen dilution, heating, acidification, digestion or combinations thereof, to enable reliable assay and specimen compatibility [McCabe et al., 1972; Martinez et al., 1973; Stumacher et al., 1973; Feldman and Pearson, 1974; Elin et al., 1975; Zinner and McCabe, 1976; and Gnauck et al., 2015]. In addition to biological inhibitors, anticoagulants, chemical inhibitors and temperature-induced pH changes of the biological specimen have been shown to interfere with the reliability of LAL [Gnauck et al., 2016]. Substrate inconsistency and batch-to-batch variability of pooled LAL reagents derived from wild-capture horseshoe crabs (i.e., ratio and function of the individual protein components) impact precision and compromise further standardization of assay methods, reaction kinetics, and sensitivity [Massignon et al, 1996].

Wild-capture LAL substrate variability has also been linked to many factors, including shifts in protein quality, activity from batch-to-batch, and year-to-year factors associated with environmental circumstances. Wild-capture LAL is collected during the spawning season, when horseshoe crabs are often physically depleted, malnourished, stressed, and exhausted from migration. These issues are compounded by deteriorating environmental conditions, such as warming ocean temperatures and acidification, pollutants and biotic stressors, habitat destruction from coastal development and dredging, and reductions in overall food availability [Wallace et al., 2014; Mattei et al., 2015]. Assessments of horseshoe crab (and many ocean species) reveal wide-ranging population declines in terms of counts, as well as health, mating behavior, movement patterns, and physical size [Chabot and Watson, 2010; Smith et al., 2017, Krisfalusi-Gannon et al., 2018; Mattei et al., 2022].

The industrial process for wild-capture horseshoe crab bleeding and LAL collection is also taxing to animal health and widely considered by scientists and environmental agencies as inhumane [Henry and Wheatly, 1992; Towle and Henry, 2003; Hurton and Berkson, 2006; Allender et al., 2010; Leschen and Correia, 2010; Coates et al., 2012; Anderson et al., 2013]. Despite years of such concerns, daily harvest limits and regulations are still set in cubic feet and based on the total volume of the bed of a typical pickup truck [Flood, 2023]. Wild-capture LAL specimen collection further requires animal transportation to offshore facilities that result in extended ocean habitat extraction, potential exposure to an array of deleterious conditions, and consequent hypoxia for 24-72 hours. The process and its associated stressors drive an estimated mortality rate of some 30% annually [Smith et al., 2020], posing increasing risks of extinction. From a material perspective, wild-capture collection from a weakened and stressed animal can impact LAL substrate quality and consistency. Further disparities arise when combining materials from randomly captured healthy and compromised animals from different regions, amplifying batch-to-batch variability. Wild-capture LAL has nonetheless met industry BET requirements to confirm the sterility of drugs, devices and water. Notably, such testing is typically performed indirectly on pharmaceutical-grade aqueous eluates, rather than the tested source materials, making the process significantly less demanding than assessing complex biological specimens.

Direct detection of infectious agents, as well as early specimen screening, with significant time and cost savings relative to blood culture and PCR, respectively, could therefore address urgent medical needs. Clinical benefits would include appropriate, timely patient antibiotic therapy, institutional safety from pathogen surveillance in healthcare settings, and to slow rising AMR trends. In the parent invention, the pathogen detection and AST assay were described using an aquaculture-derived smeLAL reagent that provided timely diagnostic results and therapeutic guidance. This continuation-in-part applies broader utility to aquaculture-derived smeLAL and hemolymph components. By overcoming the barriers to LAL use in biological specimens, the smeLAL aquaculture advancement of the parent invention leverages some 450 years of horseshoe crab evolution that enabled the species to survive an onslaught of diverse pathogens [Tanacredi, 2001]. In a different utility, this continuation-in-part teaches the biologic and therapeutic use of an smeLAL and hemolymph composition derived from aquaculture horseshoe crabs for broad-spectrum antimicrobial intervention, especially in patients that have progressed into advanced stages of sepsis.

Conventional sepsis care pathways rely on empiric administration of broad-spectrum antibiotics in nearly 67% of suspected sepsis patients, whereas use has been found to be warranted in only 13% of cases, significantly contributing to patient organ damage and the rise of AMR pathogens [Rhee et al., 2020]. In practice, antibiotic treatment is often initiated based on patient symptoms and prior to obtaining AST results, typically within 1-3 hours after blood cultures are drawn [Sterling et al., 2015]. Taken together, the parent patent and this continuation-in-part combine to establish a time-zero intervention by providing sepsis diagnosis and classification of circulating pathogens that, otherwise untreated or when treatment is delayed, can lead to morbid outcomes due to organ clearance limitations, wherein both the (1) pathogens and (2) required antibiotic concentrations can become toxic. The crucial period following a sepsis diagnosis is thus marked by rapid pathogen replication and accumulation, which can otherwise overwhelm biological defenses.

During the COVID-19 pandemic, evidence of two or more species of blood-borne pathogens, especially related to hospital acquired infections (HAIs) and interventions (i.e., mechanical ventilators, dispensing trollies, and bed units) proved catastrophic in 50% of the morbidity attributed to the pandemic [Ritter et al., 2021]. This continuation-in-part is directed at an antimicrobial, marine-derived biologic. Administration of the proposed biologic may be directed using traditional hospital management scoring systems that evaluate the severity of patient illness and predict patient outcomes. The broad-spectrum marine biologic provides comprehensive antimicrobial action beyond traditional antibiotics and notably emerging “superbugs.” These AMR pathogen strains represent the most significant global challenge of our time, claiming 1.27 million lives in 2019 and by 2050, are projected to cause 10 million deaths worldwide per year with an economic impact of upwards of 100 trillion USD. A 2023 Lancet article described the magnitude of AMR, whereby one in three patients at a Hangzhou, China, intensive care unit (ICU) became infected with a highly virulent superbug, Carbapenem-resistant Acinetobacter baumannii (CRAB), which was introduced to the ICU through patient admissions, which then continued to spread through airflow, staff contact, shared equipment, and plumbing [Doughty et al., 2023].

Two traditional clinical scoring tools, the Sequential Organ Failure Assessment (SOFA) and Acute Physiology and Chronic Health Evaluation II (APACHE II), are widely used for management of critically ill patients to inform treatment decisions. The SOFA metrics are used to evaluate the integrity of six key systems: respiratory, cardiovascular, hepatic, renal, neurological, and hematological. The score ranges from 0 to 24, with higher scores indicating reduced organ viability. The SOFA assessment is in turn used to estimate mortality risks, ranging from less than 10% for scores of 0-5, to greater than 90% for scores of 21 or higher (Table 1). Alternatively, the APACHE II assessment estimates the risk of mortality in critically ill patients, including those with sepsis. Calculated within 24 hours of hospital admission, the APACHE II score is used to guide clinical decision-making, such as needed ICU admission. The score is based on physiological variables including temperature, blood pressure, pulse, respiratory rate, arterial pH, sodium, potassium, creatinine, hematocrit, white blood cell count, Glasgow Coma Scale score, and age. The score ranges from 0 to 71, with higher scores indicating more severe illness and a higher risk of mortality (Table 2). It would follow that both scoring systems could be used to establish a framework for appropriate administration of the proposed marine biological broad spectrum therapeutic derived from antimicrobial components, smeLAL and hemolymph.

TABLE 1
Mortality Rates, Patient Condition and Risk Information by SOFA Score.

| SOFA Score | Mortality Rate | Patient Condition | Risk Information |
|---|---|---|---|
| 0-5 | <10% | Minimal dysfunction | Low risk of mortality |
| 6-9 | ~20% | Moderate dysfunction | Moderate risk of mortality |
| 10-13 | ~40% | Significant dysfunction | High risk of mortality |
| 14-17 | ~60% | Severe dysfunction | Very high risk of mortality |
| 18-20 | ~80% | Extremely severe dysfunction | Extremely high risk of mortality |
| 21+ | >90% | Organ failure | Very high risk of mortality |

TABLE 2
Mortality Rates, Patient Condition and Risk Information by APACHE II Score.

| APACHE II Score | Mortality Rate | Patient Condition | Risk Information |
|---|---|---|---|
| 0-4 | <3% | Minor illness | Low risk of mortality |
| 5-9 | ~6% | Mild to moderate illness | Low to moderate risk of mortality |
| 10-14 | ~15% | Moderate to severe illness | Moderate risk of mortality |
| 15-19 | ~25% | Severe illness | High risk of mortality |
| 20-24 | ~40% | Very severe illness | Very high risk of mortality |
| 25-29 | ~55% | Extremely severe illness | Extremely high risk of mortality |
| 30-34 | ~70% | Critical illness | Critical risk of mortality |
| 35-39 | ~85% | Extremely critical illness | Extremely critical risk of mortality |
| 40-44 | ~95% | Grave illness | Grave risk of mortality |
| 45+ | >95% | Extremely grave illness | Extremely grave risk of mortality |

The use of marine, terrestrial and bacterial-derived biological therapeutics have led to treatments for a range of diseases and have provided novel options for clinicians and patients. For example, cetuximab and brentuximab are marine-derived biologics that were developed as monoclonal antibodies for colorectal cancer treatment and non-Hodgkin's lymphoma, respectively [Cappello and Nieri, 2021]. The aquatic snail-derived peptide, ziconotide, has also been used medicinally as a pain reliever [Safavi-Hemami et al., 2019]. Keyhole Limpet Hemocyanin (KLH) is a large, oxygen-carrying protein derived from the marine mollusk, Megathura crenulate, which has gained medical significance as an immunostimulant and carrier protein for vaccines and immunotherapies [Harris and Markl, 1999]. More commonly, these biologics, such as mammalian insulin and erythropoietin, have been used to treat diabetes and anemia, respectively. A bacteria-derived biologic, streptokinase, has also been used to treat thrombosis [Vaughan et al., 1991]. Whereas recombinant endotoxin neutralizing protein (rENP) has also been used for the treatment of septic shock [Wainwright, 1991]. Notably, marine-derived rENP material (5 to 10 mg/kg) was evaluated in a rabbit study and found to be immunogenic, however without otherwise deleterious effects [Wainwright, 1991].

The primary embodiment of this invention comprises native defensive molecules isolated from aquaculture horseshoe crab smeLAL and hemolymph. A single treatment is thus expected to be proven safe for use as a critical care intervention regardless of cross-species immunogenicity. However, immunoglobulin levels decline over time and depend on several factors such as the strength of the initial response, the nature of the antigen, and the specific cells and molecules involved in the immune response. Should the need for a second injection arise, appropriate clinical parameters and oversight should be weighed against potential antigenicity concerns. Conversely, as was established with Rh immune globulin, a corresponding adjuvant immunotherapy may be envisioned and warranted to ensure repeated use, given the ultimate potential efficacy of this therapy.

Importantly, on first exposure to any new antigen, the normal immune response can take several hours to elicit inflammation and consequent symptoms. However, in septic shock, the immune system is often overwhelmed by a pathogenic challenge, leading to organ damage and increase susceptibility to further infection. Furthermore, septic shock can cause a decrease in the number and function of immune cells, including T-cells. B-cells, and neutrophils, which are associated with the immune response and production of antibodies. These factors suggest a robust therapeutic window, which could be extended indefinitely with an adjunctive use of passive smeLAL immunoglobulin.

### BRIEF DESCRIPTION OF THE INVENTION

This continuation-in-part application teaches new and broader therapeutic utility of sustainable, aquaculture-derived, modified, and enhanced LAL (smeLAL) as a biologic with ancient defense and antimicrobial properties. The parent application described an smeLAL-based diagnostic testing method for clinical screening, surveillance and diagnoses, and to perform antimicrobial susceptibility testing (AST) of small volumes of biologic specimens, regardless of opacity. The aquaculture innovations afford the smeLAL greater reactivity than that of wild-capture derived materials [Tinker-Kulberg et al., 2020] for utility in early diagnosis of infection in a clinical specimen prior to the onset of septicemia or sepsis in advance of the release of acute phase reactants and changes in blood pressure or febrile markers. The proposed invention overcomes earlier LAL substrate issues associated with wild-capture reagents for diagnostic use in biological specimens.

The smeLAL described in the parent showed greater protein density, post-translational protein modifications, allow standardization consistent with respect to reactivity, and provide functionality for clinical endpoint detection of a pathogen in biological specimens. The smeLAL material can be quality controlled to meet clinical reliability standards, maintain batch-to-batch consistency, eliminate wild-capture species impacts, and facilitate scaling for industrial and clinical applications. As such, it would be obvious to someone skilled in the art that smeLAL would be suitable as a replacement of wild-capture LAL in BET.

The performance characteristics of smeLAL in the parent invention are principally related to a profound plasticity of native protein expression in domesticated aquaculture horseshoe crabs. In an environment free of documented stressors associated with current wild-capture practices, scientifically engineered nutrition and management, including oxygenation monitoring and optimization, allows for low-impact protein collection from a healthy immunocompetent cohort of captive horseshoe crabs.

With sensitivity to some 70% of the pathogens that lead to sepsis, the smeLAL horseshoe crab blood components. i.e., coagulin, pro-clotting enzymes, Factor C. Factor B and Factor G, are more concentrated, reactive and compatible with biological specimens compared to those isolated from wild-capture horseshoe crabs [Tinker-Kulberg et al., 2020].

In the primary embodiment of this proposed invention, an smeLAL and optional hemolymph composition is isolated from aquaculture horseshoe crabs and processed to preserve structural and functional integrity for use as an injectable broad-spectrum antimicrobial biologic. Fully funded animal and safety studies may determine routine management of or inconsequential cross-species immunological reactions, just as many traditional avian-derived vaccines have been adopted worldwide [Rajaram et al., 2020]. Nonetheless, the risks associated with sepsis could override moderate species reactions in cases where standard treatment strategies (e.g., antibiotics, antifungals, antivirals, and combination therapies; immunomodulatory and cytokine inhibitor therapies; and extracorporeal therapies) have failed; as such, this invention and composition may represent a last resort therapy, as with investigational agents. Alternatively, those skilled in the art may consider development of an adjuvant smeLAL immunoglobulin therapeutic for expanded utility.

In an alternative embodiment, a biologically derived antimicrobial agent may similarly be proven as a viable primary therapeutic given that atypical histamine response can be managed (e.g., epinephrine) in controlled settings, in the event of more consequential reactions in clinical studies. In contrast to synthetic antibiotics, the efficacy of polymicrobial treatments is not limited by molecular ratios or organ clearance issues, as it has an expected microbiocidal contact activity with a rapid therapeutic window. This corresponds with the use of aggressive renal clearance fluids, or dialysis, which help reduce the pathogen load in the bloodstream. It should also be noted that sepsis can impede an otherwise normal immune response to antigenic matter.

The current invention teaches the first application and use of naturally optimized biomolecules and components isolated from the smeLAL and optional hemolymph of aquaculture horseshoe crabs to establish a novel antimicrobial biologic therapy for sepsis and potentially for an array of otherwise AMR pathogens. The primary embodiment may thus be considered useful as an injectable therapy of last resort for severe sepsis cases. It would be obvious to one skilled in the art that the composition could also be useful as infusion, subcutaneous injection, topical formulation, or other delivery method, including optimization for oral administration.

### DETAILED DESCRIPTION OF THE INVENTION

The parent invention is related to an smeLAL and method for direct detection of free-circulating LPS and intact gram-negative bacteria or fungi in a small-volume, biological specimen, irrespective of sample opacity. The aquaculture aspect of the parent invention overcomes issues described in the prior art in the field using LAL substrates that failed to reliably detect the presence of a pathogen in a biological specimen, to differentiate gram-negative bacteria from fungal pathogens, and to determine antimicrobial susceptibility of infectious agents. This continuation-in-part application teaches that the smeLAL and optional hemolymph composition isolated from aquaculture horseshoe crabs as an antimicrobial therapeutic, with particular utility in the treatment of septicemia, as well as tissue, systemic and organ infections, including those caused by emerging AMR pathogens.

In the primary embodiment of the invention, the material consisting of aquaculture derived smeLAL and optional hemolymph is collected, prepared, and subjected to a series of purification steps to concentrate the desired biologic molecules. These steps include filtration, chromatography, and other separation techniques, as appropriate with respect to the specific properties of the material. Quality control assays can be used to assess the purity and activity of the material, and adjustments may be made during the purification process. The goal of purifying aquaculture horseshoe crab smeLAL and hemolymph material for use as a human biologic is to produce a safe, effective, and highly sensitive product. The final composition would be further processed for extended shelf life using routine and specialized techniques such as cryopreservation, freeze-drying, chemical stabilization and tableting if optimized for oral administration.

The marine biologic of the present invention comprises a complex mixture of native components isolated from aquaculture horseshoe crab hemolymph. The mixture contains various proteins, enzymes, metabolites, and other molecules that synergize as a broad-spectrum antimicrobial with manifold activity to neutralize infectious pathogens.

One component of the biologic, smeLAL, is comprised of the intracellular materials of the circulating amebocyte cell. Within the amebocyte, the Large granule (L-granule) contains several coagulation factors associated with the organism's clotting response and plays a critical role in the animal's immunity [Iwanaga et al., 1998; Kawabata et al., 2003]. The key defense molecules associated with the horseshoe crab L-granule include: four serine proteases (i.e., Factor C. Factor B. Factor G and pro-clotting enzymes), as well as a large, multi-domain coagulation protein (coagulogen). In addition to their role in clotting. Factor C (123 kDa). Factor B (64 kDa), and Factor G (110 kDa) proteins provide immune modulating capabilities including antimicrobial activity from broad-spectrum phagocytosis of microbial matter of bacterial, fungal, or viral origin by circulating immune cells (i.e., macrophages). The pro-clotting enzymes and coagulogen (20 kDa) proteins play a key role in clot formation and stabilization, thereby in controlling bleeding, limiting spread of infection, and reducing the formation of abnormal clots throughout the body, which can damage and lead to organ failure. Aquaculture-derived coagulation factors in the L-granules of horseshoe crab amebocytes thus represent a pathway for sustainable production of highly concentrated material that is more reactive than that isolated from wild-capture conspecifics.

In addition to smeLAL coagulation factors, the hemolymph and cellular components of the horseshoe crab contain numerous protease inhibitors, including: Limulus intracellular coagulation inhibitors (LICI-1, 2, and 3); Limulus trypsin inhibitor (LTI); chymotrypsin inhibitor; trypsin inhibitors; Limulus endotoxin-binding protein-protease inhibitor (LEBP-PI); Limulus cystatin; and alpha-2-macroglobulin (A2M) [Agarwala et al., 1996; Miura et al., 1995; Miura et al., 1994; Armstrong, 2010; Armstrong et al., 1991; Melchior et al., 1995; Iwaki et al., 1996]. The LICI molecules have anti-inflammatory properties that can modulate the immune response and prevent excessive inflammation and tissue damage through down regulation of cytokines associated with the cytokine “storm” during sepsis. Specifically, management of interleukin-6 (IL-6), a major driver of the acute-phase inflammatory response, and tumor necrosis factor-alpha (TNF-alpha), can play a crucial role in the rate of activation of immune cells and the cytokine cascade. During sepsis, horseshoe crab protease proteins. LTI, chymotrypsin inhibitor and trypsin inhibitor, can prevent over-activation of trypsin and chymotrypsin and mitigate tissue damage, dampen the inflammatory response and diminish the overall cytokine storm. Further. LTI has direct antimicrobial effects against both gram-negative and gram-positive bacteria by inhibiting bacterial proteases that promote growth and replication while enhancing the activity of antimicrobial peptides and other immune response molecules.

Another essential molecule isolated from L-granule of smeLAL is Limulus endotoxin-binding protein-protease inhibitor (LEBP-PI), a hybrid protein comprising an endotoxin-binding protein and protease inhibitor, which synergistically binds to bacterial endotoxins and inhibits protease enzymes. During sepsis, lipopolysaccharides (LPS) trigger an overwhelming inflammatory response that typically leads to tissue and organ damage. LEBP-PI binds to LPS, neutralizing the endotoxin, thereby controlling the inflammatory response. Limulus cystatin is a cysteine protease activation inhibitor (i.e., cathepsins) as well as derivative activation of inflammatory cytokines (i.e., IL-1B and IL-18) and the breakdown of extracellular proteins. Finally, alpha-2-macroglobulin (A2M) inhibits the activity of IL-6 and TNF-alpha cytokines by forming a complex that facilitates clearance from circulation and dampening the cytokine storm. A2M also has a direct antimicrobial effect against gram-negative and gram-positive bacteria by binding to cell membrane components, which activates the complement system and fosters bacterial clearance.

The marine biologic of the present invention further comprises several antimicrobial polypeptides and single-chain peptides stored in the small and large granules (S- and L-granules). The S-granule-derived antimicrobial peptides bind to chitin but no other polysaccharides, such as cellulose, mannan, xylan, and laminarin, to interact with pathogen membranes. Another small peptide found in the L-granule, anti-LPS factor, binds to LPS and neutralizes the endotoxin, to ultimately lower the inflammatory response and endotoxin burden. Anti-LPS factor also plays a key role in inhibition of viral replication by preventing host cell entry. A serine protease. Factor D, cleaves coagulation Factor B and results in the formation of convertase, which opsonizes and lyses bacterial cells. Tachyplesins, Polyphemusins, Tachycitin, and Tachystatins are also antimicrobial peptides contained in the S-granule of amebocytes with robust activity that can rapidly neutralize and inhibit proliferation of gram-negative and gram-positive bacteria, fungi and viruses. The cationic peptides. Tachyplesin. Tachycitin, and Polyphemusins, directly target pathogens and disrupt membrane integrity to induce cell lysis and destruction. Tachyplesins also disrupt viral envelopes and prevent virion entry into host cells. The anionic peptide. Tactystatin, positively charges bacterial and fungal membranes and disrupts integrity through electrostatic interactions. Both the L- and S-granules contain molecules like mammalian neutrophil-derived defensins, but they are distinctly larger and inhibit growth and replication of gram-negative and gram-positive bacteria and fungi. By neutralizing bacterial and fungal toxins, growth inhibition, and enhancing clearance, these substances inhibit pathogens with an array of mechanisms with expected applicability in the treatment of blood borne and AMR pathogen infections.

Numerous lectins in both the plasma and granules, and several bacterial agglutinins, also interact with infectious microbes [Iwanaga et al., 1998; Kawabata et al., 2003]. Several horseshoe crab lectins (i.e., Tachylectins 1, 2, 3, 4 and 5 and Limunectin) are proteins that can bind to specific carbohydrate molecules on bacterial membranes, leading to pathogen agglutination and enhanced clearance. Horseshoe crab lectins have also demonstrated broad-spectrum antimicrobial activity against both gram-negative and gram-positive bacteria. Horseshoe crab bacterial agglutinins (i.e., T. Tridentatus Agglutinin, and Limulus 18-kDa Agglutination-aggregation Factor) are antibodies that bind to specific antigens on the surface of the bacteria, leading to agglutination and enhanced clearance. Horseshoe crab bacterial agglutinins, such as Limulus anti-LPS factor (LALF), have also demonstrated antibacterial activity against a range of gram-negative bacteria, including those that have been increasingly resistant to antibiotics.

The hemolymph of horseshoe crabs contains two immunomodulatory proteins, Galactose-binding protein (GBP) and protein A binding protein (PAP). GBP can bind to carbohydrates on bacterial cells and enhance phagocytosis by neutrophils and macrophages. Whereas PAP binds to the IgG antibodies, inhibits the cytokine production associated with the cytokine storm, and enhances bacterial clearance by activating immune cells and stimulating the release of antimicrobial molecules (i.e., reactive oxygen species and nitric oxide).

Biologically, C-reactive protein (CRP) is essential to the first line of defense against infections. The horseshoe crab hemolymph contains a class of CRP, or Limulin, that exhibits cytolytic and opsonic activities against foreign cells and bears structural similarity to the complement system in mammals [Iwaki et al., 1999]. Transglutaminase (TGase) found in the cytosol of horseshoe crab amebocytes also assists in cross-linking the coagulin gel, in the immobilization of invasive microbes, and stabilization of the blood clot to limit spread of infection. In sepsis, the immune response can lead to over-activation of the clotting system, which can cause disseminated intravascular coagulation (DIC), a condition in which blood clots form throughout the body, leading to organ damage and risk of failure. By promoting the formation of stable clots. TGase helps prevent such thrombotic anomalies without interfering with normal clotting in response to injury or local infection.

As the predominant protein component (i.e., 50% to 90%) of invertebrate hemolymph, hemocyanin is a multi-functional protein that provides oxygen transport and host immunity. Its oxygen binding affinity is modulated by protons from various organic ions (e.g., calcium, sodium, chloride, potassium, magnesium, lactate, urate, etc.) with optimal binding at or around physiological pH and across a broad temperature range (4 to 37° C.). In contrast to hemoglobin dependence on negative allosteric effectors for oxygen diffusion, hemocyanin can passively release oxygen with a gradient that optimizes uniform delivery, without requiring external allosteric interactions. During sepsis, the immune response can lead to hypoxia, which can contribute to organ dysfunction and failure. Given the primary function to transport oxygen to respiring tissues, hemocyanin contains di-cupric groups that reversibly bind molecular oxygen [van Holden et al., 2001] and play a dual role in host immunity. Immunologically, hemocyanin is also capable of converting into phenoloxidase-like (PO) enzymes upon physical disruption of the structural motifs surround central copper molecules. These POs play vital roles in a broad-based response to pathogens [Coates et al., 2014]; however, intact hemocyanin demonstrates greater activity than POs against bacteria and fungi.

In a primary embodiment of the present invention, the complex mixture of proteins, enzymes, and other biomolecules derived from the smeLAL and hemolymph of aquaculture horseshoe crabs is subjected to a purification process to ensure maximum yield, purity, activity and material safety. In one aspect of the present invention, a multi-step process is used to purify horseshoe crab proteins and biomolecules. The process involves sterile isolation of the material from aquaculture horseshoe crabs through the pericardial membrane and clarifying the composition through centrifugation, dialysis, extraction, and filtration. Subsequently, traditional fractionation techniques (i.e., solvent extraction) may be used to separate the hemolymph components based on solubility or hydrophobicity. The individual fractions may then be concentrated using, but not limited to, methods such as ammonium sulfate precipitation, and purified using various chromatographic techniques (e.g., ion-exchange, size-exclusion, affinity, and hydrophobic interaction chromatography). Hemolymph contains various proteins, biomolecules, such as the oxygen-carrying molecule hemocyanin, and smeLAL, thus a combination of purification techniques and multiple rounds of chromatography may be required for optimal therapeutic embodiments.

In the primary embodiment, each fraction is then subjected to sequential chromatographic columns, including ion exchange, hydrophobic interaction, size exclusion, and affinity chromatography to separate components based on charge, hydrophobicity, size, or specific binding properties. Additional purification steps and buffer exchanges can be performed for further processing or storage. The purified composition is then subjected ultrafiltration and diafiltration for concentration and buffer exchange. Analytical techniques, including SDS-PAGE. Western blot, mass spectrometry, and enzyme activity assays, are used to monitor the separation process and identify components in each fraction. The purified proteins are would then be sterile filtered, formulated into the desired final configuration, and packaged for distribution. Similarly, each purification process may be performed specific to the protein of interest to ensure optimal yield, purity, and activity, while meeting regulatory requirements for safety, efficacy, and quality. The process may be used to isolate specific components for a desired application. It would be obvious to one skilled in the art that one or many of the components isolated and purified may be formulated into a given biological composition.

In an exemplary composition of a preferred embodiment of the invention, the processed horseshoe crab biomolecules are combined with carriers or excipients routinely used in pharmaceutical formulations to improve stability, solubility, bioavailability, delivery and/or to manage immunogenicity. Such substances may include buffering components, such as sterile isotonic solutions of sodium chloride in water (i.e., saline solution), phosphate buffered saline, tris-buffered saline, and/or HEPES-buffered saline to maintain pH and ionic strength of the solution. Similarly, sterile, pyrogen-free water for injection common to parenteral drug formulations would represent another suitable formulation. Alternatively, stabilizers, like sugars (e.g., dextrose, sucrose, trehalose), amino acids (e.g., glycine, L-arginine) and polyols (e.g., glycerol, mannitol, sorbitol) that protect sensitive proteins and molecules may be included to prevent aggregation, denaturation, and degradation. The use of non-ionic surfactants, such as polysorbate-20 and polysorbate 80, are common additives for preventing aggregation, improving solubility and reducing adsorption to surfaces. In alternative embodiments, the inclusion of antioxidant substances (i.e., ascorbic acid or sodium metabisulfite) may be used to prevent oxidative degradation. Other formulations may utilize chelating agents (i.e., EDTA or citrate) to prevent metal-catalyzed oxidation and aggregation. Lastly, the use of carrier proteins (i.e., human and other albumin sources) or biocompatible and biodegradable polymers (e.g., polyethylene glycol, PEG), may be used to improve stability and solubility while improving bioavailability and reducing immunogenicity of the injectable and/or alternative therapeutic compositions.

The formulation of the injectable and other therapeutic compositions is not limited to these substances and would be obvious to someone skilled in the art to include alternative pharmaceutical formulations or combinations thereof. A suitable formulation may also comprise a pharmaceutical-grade preservative, such as but not limited to, benzyl alcohol. It would be further considered obvious to someone skilled in the art that the purified smeLAL and hemolymph constituents may be specifically combined or individually formulated into the final injectable or other composition, depending on the administration method. Specifically, the purified smeLAL and hemolymph constituents would also be expected to have routine utility in non-injectable formulations, such as topical gels, sprays, creams, inhalants, drops, and tablets if optimized for oral administration.

What is claimed:

1. A broad-spectrum antimicrobial therapeutic composition with antimicrobial activity to gram-negative bacterial, gram-positive bacterial, fungal and viral pathogens that is useful for treating infections, including septicemia, severe sepsis, and septic shock, comprising:
a. purified aquaculture-derived horseshoe crab smeLAL,
b. purified aquaculture-derived horseshoe crab hemolymph and/or its biomolecular components;
c. purified aquaculture-derived horseshoe crab hemocyanin;
wherein the composition includes a suitable concentration of a pharmaceutical-grade carrier or excipient.

2. The broad-spectrum antimicrobial therapeutic composition of claim 1 that is injectable into a patient.

3. The broad-spectrum antimicrobial therapeutic composition of claim 1, that is formulated as an aerosol, cream, droplet suspension, emulsion, ethosome, exosome, foam, gel, hydrogel, liposome, micelle, ointment, spray or another suitable therapeutic vehicle.

4. The broad-spectrum antimicrobial therapeutic composition of claim 1 for use as a topical or external agent.

5. The broad-spectrum antimicrobial therapeutic composition of claim 1 for use as an ingestible formulation for oral administration.

6. The broad-spectrum antimicrobial therapeutic composition of claim 1, that is used as a last-resort therapy for severe sepsis or septic shock.

7. The broad-spectrum antimicrobial therapeutic composition of claim 1, that is used as a primary treatment for bloodstream infections, including septicemia caused by antimicrobial resistant pathogens.

8. The pharmaceutical-grade carriers in claim 1, wherein the pharmaceutical-grade carriers comprise one or more of the following: saline solution, phosphate buffered saline, tris-buffered saline, HEPES-buffered, pyrogen free water for injection, carrier proteins and biocompatible, biodegradable polymers.

9. The pharmaceutical-grade excipients of claim 1, wherein the pharmaceutical-grade excipients include one or more of the following: sugars, amino acids, polyols, non-ionic surfactants, antioxidant substances, chelating agents, and preservatives.

10. The patient of claim 1, wherein the patient includes: humans; veterinary animals, such as companion animals; and agricultural animals, such as livestock and poultry.
